(19) United States
(12) Patent Application Publication
(10) Pub. No.: US 2026/0333891 A1
(43) Pub. Date: Oct. 22, 2026

(54) SEMICONDUCTOR DEVICE, OPTOELECTRONIC LOGIC GATE, PHOTODETECTOR, AND METHOD FOR MANUFACTURING SEMICONDUCTOR DEVICE

(71) Applicant: Fujitsu Limited, Kawasaki-shi (JP)

(72) Inventors: Maciej Franciszek MATYS, Atsugi (JP); Atsushi YAMADA, Hiratsuka (JP); Toshihiro OHKI, Hadano (JP)

(73) Assignee: Fujitsu Limited, Kawasaki-shi (JP)

(21) Appl. No.: 19/646,684

(22) Filed: Apr. 14, 2026

(30) Foreign Application Priority Data
Apr. 16, 2025 (JP) 2025-067645

(51) Int. Cl. H10F 30/28; G01N 33/00; G02F 3/00; H10F 71/00; H10F 77/124; H10F 77/14

(52) U.S. Cl. CPC H10F 30/2877; H10F 71/1278; H10F 77/1246; H10F 77/146; G01N 33/0027; G02F 3/00

(57) ABSTRACT

A semiconductor device includes a first barrier layer; a second barrier layer disposed on the first barrier layer; a channel layer disposed on the second barrier layer; a first ohmic electrode electrically connected to the channel layer; and a second ohmic electrode separated from the first ohmic electrode and electrically connected to the channel layer, wherein the first barrier layer has a superlattice structure including a first nitride semiconductor layer containing Al and a second nitride semiconductor layer containing Ga, the second barrier layer includes a third nitride semiconductor layer containing Al and Ga, the channel layer includes a fourth nitride semiconductor layer containing Ga, and a surface of the second barrier layer facing the channel layer has nitrogen polarity.

### CROSS-REFERENCE TO RELATED APPLICATIONS

The present application is based upon and claims the benefit of priority from the prior Japanese Patent Application No. 2025-067645 filed on Apr. 16, 2025, with the Japanese Patent Office, the entire contents of which are incorporated herein by reference.

### FIELD

The present disclosure relates to a semiconductor device, an optoelectronic logic gate, a photodetector, and a method for manufacturing a semiconductor device.

### BACKGROUND

Conventionally, negative photoconductivity has been reported in graphene and crystals having a perovskite structure.

[Related-Art Documents]

### Patent Documents

### Patent Documents

Patent Document 1: Japanese Laid-open Patent Publication No. H04-170526
Patent Document 2: U.S. Pat. No. 5,451,767
Patent Document 3: Japanese Laid-open Patent Publication No. H04-110834
Patent Document 4: US Patent Application Publication No. 2016/0274301

### Non-Patent Documents

Non-Patent Document 1: Biswas C, Gunes F, Duong D L, Lim S C, Jeong M S, Pribat D and Lee Y H 2011 Nano Lett. 11 4682
Non-Patent Document 2: M. A. Haque, J.-L. Li, A. L. Abdelhady, M. I. Saidaminov, D. Baran, O. M. Bakr, S.-H. Wei, T. Wu, Adv. Optical Mater. 2019, 7, 1900865
Non-Patent Document 3: Wang, Y. et al. ACS Nano 12, 9513-9520 (2018)

### SUMMARY

According to an aspect of embodiments of the present disclosure, a semiconductor device is provided. The semiconductor device includes a first barrier layer;
a second barrier layer disposed on the first barrier layer;
a channel layer disposed on the second barrier layer;
a first ohmic electrode electrically connected to the channel layer; and
a second ohmic electrode separated from the first ohmic electrode and electrically connected to the channel layer, wherein
the first barrier layer has a superlattice structure including a first nitride semiconductor layer containing Al and a second nitride semiconductor layer containing Ga,
the second barrier layer includes a third nitride semiconductor layer containing Al and Ga,
the channel layer includes a fourth nitride semiconductor layer containing Ga, and
a surface of the second barrier layer facing the channel layer has nitrogen polarity.

The object and advantages of the embodiment will be realized and attained by means of the elements and combinations particularly pointed out in the claims.

It is to be understood that both the foregoing general description and the following detailed description are exemplary and explanatory and are not restrictive of the invention, as claimed.

### BRIEF DESCRIPTION OF DRAWINGS

FIG. 1 is a cross-sectional view illustrating a semiconductor device according to a first embodiment.

FIG. 2 is a cross-sectional view illustrating a first barrier layer.

FIG. 3 is a diagram illustrating a band structure of the semiconductor device according to the first embodiment.

FIG. 4 is a cross-sectional view illustrating a method for manufacturing the semiconductor device according to the first embodiment.

FIG. 5 is a graph illustrating change in current over time when light having a wavelength of 430 nm is applied in a first experiment.

FIG. 6 is a graph illustrating change in current over time when light having a wavelength of 610 nm is applied in the first experiment.

FIG. 7 is a graph illustrating change in current over time when light having a wavelength of 990 nm is applied in the first experiment.

FIG. 8 is a graph illustrating change in current over time when light having a wavelength of 990 nm is applied in a second experiment.

FIG. 9 is a diagram illustrating a photodetector according to a second embodiment.

FIG. 10 is a diagram illustrating an optoelectronic logic gate according to a third embodiment.

### DESCRIPTION OF EMBODIMENTS

Depending on the application, a new semiconductor device capable of exhibiting negative photoconductivity is desired.

The object of the present disclosure is to provide a new semiconductor device capable of exhibiting negative photoconductivity, an optoelectronic logic gate, a photodetector, and a method for manufacturing a semiconductor device.

According to the present disclosure, negative photoconductivity can be exhibited.

Hereinafter, embodiments of the present disclosure will be specifically described with reference to the accompanying drawings. Note that, in the present specification and drawings, components having substantially the same functional configuration are denoted by the same reference numerals to omit redundant description.

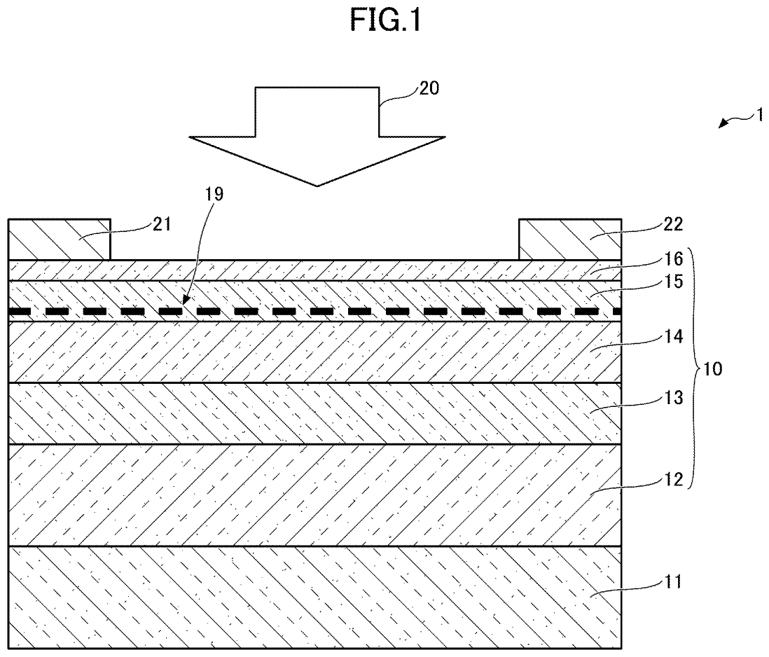

(First Embodiment) The first embodiment will be described. The first embodiment relates to a semiconductor device. FIG. 1 is a cross-sectional view illustrating the semiconductor device according to the first embodiment.

The semiconductor device 1 according to the first embodiment includes, as illustrated in FIG. 1, a substrate 11 and a nitride semiconductor stacked structure 10 provided above the substrate 11. The nitride semiconductor stacked structure 10 includes a buffer layer 12, a first barrier layer 13, a second barrier layer 14, a channel layer 15, and a cap layer 16. The buffer layer 12 is disposed on the substrate 11. The first barrier layer 13 is disposed on the buffer layer 12. The second barrier layer 14 is disposed on the first barrier layer 13. The channel layer 15 is disposed on the second barrier layer 14. The cap layer 16 is disposed on the channel layer 15.

The substrate 11 is, for example, a SiC substrate, a GaN substrate, or a sapphire substrate. When the substrate 11 is a SiC substrate, the upper surface of the substrate 11 has carbon (C) polarity. When the substrate 11 is a GaN substrate, the upper surface of the substrate 11 has nitrogen (N) polarity. When the substrate 11 is a sapphire substrate, the upper surface of the substrate 11 has an off-cut angle. The magnitude of the off-cut angle is, for example, about 4°. The buffer layer 12 has an AlN layer, a GaN layer, an AlGAN layer, or any combination thereof. The Al composition in the AlGaN layer may be constant or may vary. For example, when the buffer layer 12 has a GaN layer and an AlGaN layer thereon, the Al composition in the AlGaN layer may increase with distance from the GaN layer. The thickness of the buffer layer 12 is, for example, about 1 μm.

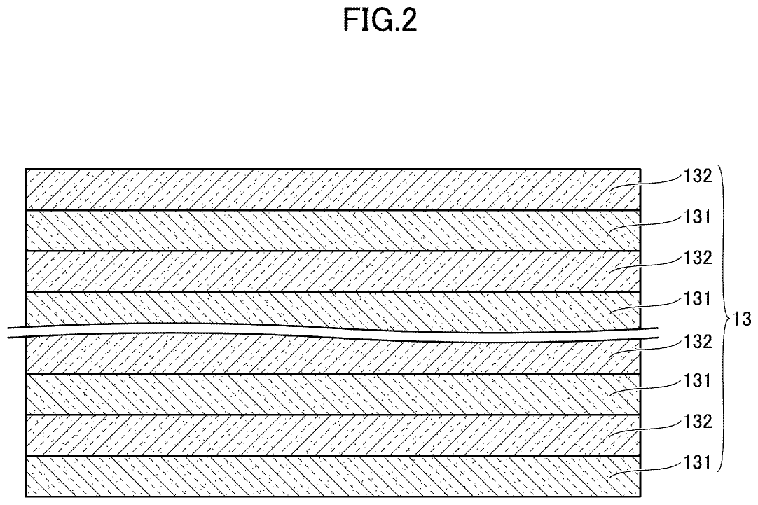

FIG. 2 is a cross-sectional view illustrating the first barrier layer 13. The first barrier layer 13 includes a superlattice structure having a first nitride semiconductor layer 131 containing Al and a second nitride semiconductor layer 132 containing Ga. That is, as illustrated in FIG. 2, the first barrier layer 13 has one or more pairs of the first nitride semiconductor layer 131 and the second nitride semiconductor layer 132. When the first barrier layer 13 has two or more pairs of the first nitride semiconductor layer 131 and the second nitride semiconductor layer 132, the first nitride semiconductor layer 131 and the second nitride semiconductor layer 132 are alternately arranged. The first nitride semiconductor layer 131 is, for example, an AlN layer having a thickness of 1 nm or more and 10 nm or less. The second nitride semiconductor layer 132 is, for example, a GaN layer having a thickness of 1 nm or more and 30 nm or less. The thickness of the second nitride semiconductor layer 132 may be equal to, greater than, or less than the thickness of the first nitride semiconductor layer 131. In one example, the first nitride semiconductor layer 131 is an AlN layer having a thickness of 5 nm, and the second nitride semiconductor layer 132 is a GaN layer having a thickness of 10 nm.

The second barrier layer 14 has a third nitride semiconductor layer containing Al and Ga. The third nitride semiconductor layer is, for example, an AlₓGa₁₋ₓN layer (0<x<1) having a thickness of 5 nm or more and 40 nm or less. The value of x is, for example, 0.10 or more and 0.50 or less. The channel layer 15 has a fourth nitride semiconductor layer containing Ga. The fourth nitride semiconductor layer is, for example, a GaN layer having a thickness of 10 nm or more and 30 nm or less. The cap layer 16 has an AlN layer, a GaN layer, an AlGaN layer, or any combination thereof. The thickness of the cap layer 16 is, for example, 1 nm or more and 5 nm or less. A two-dimensional electron gas (2DEG) 19 exists near the lower surface of the channel layer 15. In one example, the second barrier layer 14 is an Al_{0.3}Ga_{0.7}N layer having a thickness of 30 nm, the channel layer 15 is a GaN layer having a thickness of 20 nm, and the cap layer 16 is a stacked body of an Al_{0.4}Ga_{0.6}N layer having a thickness of 3 nm and a GaN layer having a thickness of 2 nm thereon.

The semiconductor device 1 has a first ohmic electrode 21 and a second ohmic electrode 22. The first ohmic electrode 21 and the second ohmic electrode 22 are disposed on the nitride semiconductor stacked structure 10. The first ohmic electrode 21 and the second ohmic electrode 22 are separated from each other. The first ohmic electrode 21 and the second ohmic electrode 22 are in contact with the nitride semiconductor stacked structure 10 and are electrically connected to the channel layer 15. In one example, each of the first ohmic electrode 21 and the second ohmic electrode 22 is a stacked body of a titanium (Ti) film and an aluminum (Al) film thereon.

In the semiconductor device 1 configured as described above, a current flows between the first ohmic electrode 21 and the second ohmic electrode 22 through the 2DEG 19 in accordance with a voltage applied between the first ohmic electrode 21 and the second ohmic electrode 22. At this time, when light 20 is applied onto the semiconductor device 1, the semiconductor device 1 exhibits photoconductivity depending on the wavelength of the light 20. For example, when light having a wavelength of 800 nm or shorter is applied as the light 20, the semiconductor device 1 exhibits positive photoconductivity, the mobility of electrons in the 2DEG 19 increases, and the current increases. On the other hand, when light having a wavelength longer than 800 nm is applied as the light 20, the semiconductor device 1 exhibits negative photoconductivity, the mobility of electrons in the 2DEG 19 decreases, and the current decreases.

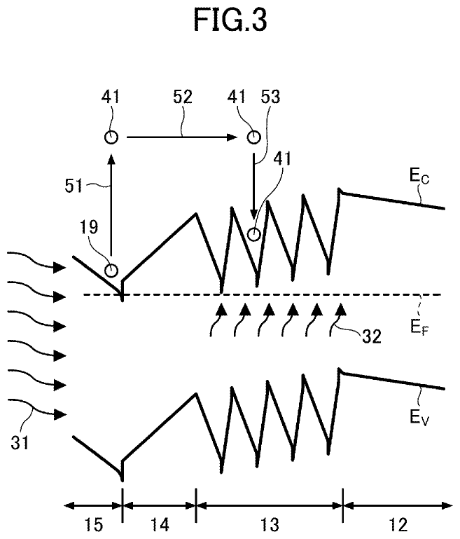

Here, the negative photoconductivity exhibited by the semiconductor device 1 will be described. FIG. 3 is a diagram illustrating the band structure of the semiconductor device 1. In FIG. 3, Er denotes the Fermi level, E_{C} denotes the energy of the lower edge of the conduction band, and Ev denotes the energy of the upper edge of the valence band.

When light having a wavelength longer than 800 nm, for example infrared light 31, is applied onto the semiconductor device 1, hot electrons 41 are excited from the 2DEG 19 (process 51). The excited hot electrons 41 diffuse through the second barrier layer 14 to the first barrier layer 13 having the superlattice structure (process 52). Then, the hot electrons 41 that have diffused to the first barrier layer 13 are trapped in the first barrier layer 13 (process 53). As a result, the energy possessed by the hot electrons 41 is transferred as heat 32 to the first barrier layer 13, and since the thermal conductivity of the first barrier layer 13 having the superlattice structure is low, the temperature in the vicinity of the interface between the first nitride semiconductor layer 131 and the second nitride semiconductor layer 132 increases. Consequently, the mobility of electrons in the 2DEG 19 decreases, and the current decreases.

The number of pairs of the first nitride semiconductor layer 131 and the second nitride semiconductor layer 132 included in the superlattice structure of the first barrier layer 13 is not limited, but it is preferable that the superlattice structure has four or more pairs of the first nitride semiconductor layer 131 and the second nitride semiconductor layer 132. This is because when the superlattice structure has four or more pairs of the first nitride semiconductor layer 131 and the second nitride semiconductor layer 132, positive photoconductivity and negative photoconductivity tend to be more pronounced.

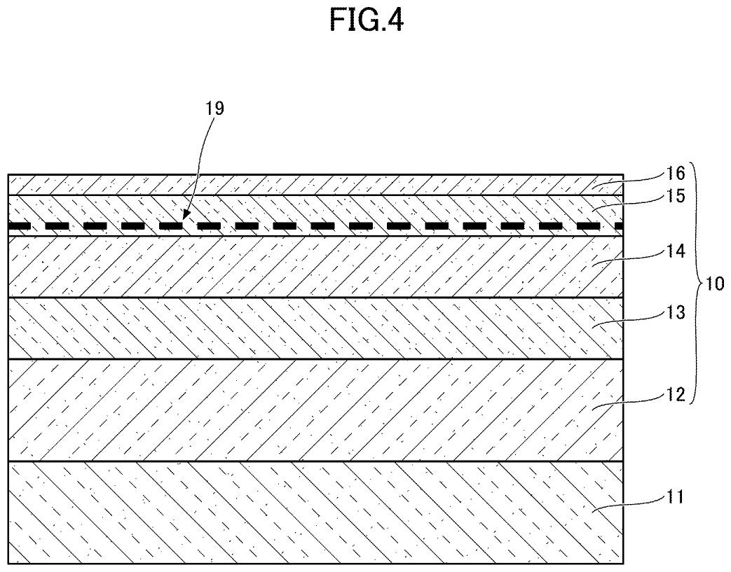

Next, a method for manufacturing the semiconductor device 1 according to the first embodiment will be described. FIG. 4 is a cross-sectional view illustrating the method for manufacturing the semiconductor device according to the first embodiment.

First, as illustrated in FIG. 4, the nitride semiconductor stacked structure 10 is formed on the substrate 11. In forming the nitride semiconductor stacked structure 10, the buffer layer 12 is formed on the substrate 11, the first barrier layer 13 is formed on the buffer layer 12, and the second barrier layer 14 is formed on the first barrier layer 13. Further, the channel layer 15 is formed on the second barrier layer 14, and the cap layer 16 is formed on the channel layer 15. The nitride semiconductor stacked structure 10 can be formed, for example, by metal organic chemical vapor deposition (MOCVD). A 2DEG 19 is generated near the lower surface of the channel layer 15.

Next, the first ohmic electrode 21 and the second ohmic electrode 22 are formed on the nitride semiconductor stacked structure 10 (see FIG. 1). The first ohmic electrode 21 and the second ohmic electrode 22 can be formed, for example, by evaporation and lift-off.

In this manner, the semiconductor device 1 according to the first embodiment can be manufactured.

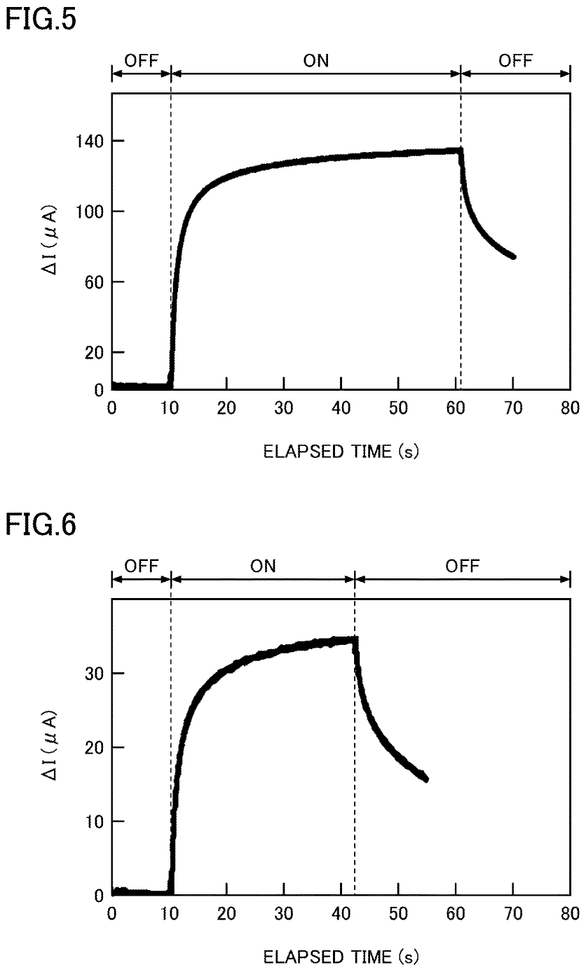

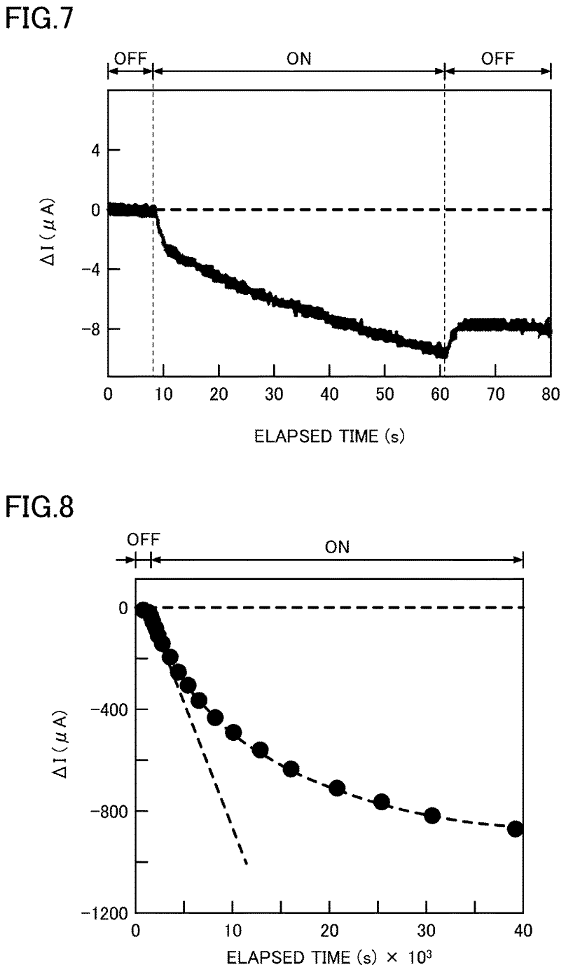

Here, experiments conducted by the present inventor relating to the first embodiment will be described.

[First Experiment] In the first experiment, an experimental semiconductor device was fabricated in accordance with the first embodiment, and the photoconductivity of this semiconductor device was confirmed. In this semiconductor device, the number of pairs of the first nitride semiconductor layer 131 and the second nitride semiconductor layer 132 included in the superlattice structure of the first barrier layer 13 was set to four. A predetermined voltage was applied between the first ohmic electrode 21 and the second ohmic electrode 22, and changes in current over time were measured when three types of light having wavelengths of 430 nm, 610 nm, and 990 nm were applied. The results are illustrated in FIGS. 5 to 7. FIG. 5 is a graph illustrating the change in current over time when light having a wavelength of 430 nm is applied in the first experiment. FIG. 6 is a graph illustrating the change in current over time when light having a wavelength of 610 nm is applied in the first experiment. FIG. 7 is a graph illustrating the change in current over time when light having a wavelength of 990 nm is applied in the first experiment. “On” and “Off” in FIGS. 5 to 7 indicate “on” and “off” of light application onto the semiconductor device, respectively. Further, ΔI (μA) on the vertical axis represents the difference (I_{light}−I_{dark}) between the current I_{light} when light application is “on” and the current I_{dark} when light application is “off”.

As illustrated in FIGS. 5 to 7, it was confirmed that the fabricated semiconductor device exhibits positive photoconductivity when light having a wavelength of 800 nm or shorter is applied, and exhibits negative photoconductivity when light having a wavelength longer than 800 nm is applied.

[Second Experiment] In the second experiment, using the semiconductor device fabricated in the first experiment, a predetermined voltage was applied between the first ohmic electrode 21 and the second ohmic electrode 22 in the same manner as in the first experiment, and the change in current over time was measured when light having a wavelength of 990 nm was applied. However, in the second experiment, the application time of light was made longer to bring the negative photoconductivity close to a saturated state. The result is illustrated in FIG. 8. FIG. 8 is a graph illustrating the change in current over time when light having a wavelength of 990 nm is applied in the second experiment.

As illustrated in FIG. 8, the negative photoconductivity began to saturate around 5000 seconds, but was not completely saturated even after 40000 seconds. Here, Y_{PC} defined by the following Equation 1 is defined as photoconductivity yield. In Equation 1, I_{S} is the current when saturated.

Y_{PC}=|I_{S}−I_{dark}|/|I_{dark}|×100 (Equation 1)

In the example illustrated in FIG. 8, the absolute value of the current I_{dark} when light application is “off” is approximately 1 mA, and the current difference ΔI (μA) at the point after 40000 seconds have elapsed is approximately-0.85 mA. Since the current difference ΔI (μA) when saturated will be lower than-0.85 mA, the photoconductivity yield Y_{PC} in the example illustrated in FIG. 8 will be greater than 85%.

Until now, negative photoconductivity has also been reported in graphene (Non-Patent Document 1), but the photoconductivity yield Y_{PC} in graphene is at most 34%. Negative photoconductivity has also been reported in crystals having a perovskite structure of Bi-doped MAPbBr₃ (MA is CH₃NH₃) (Non-Patent Document 2), but the photoconductivity yield Y_{PC} in this material is 67%.

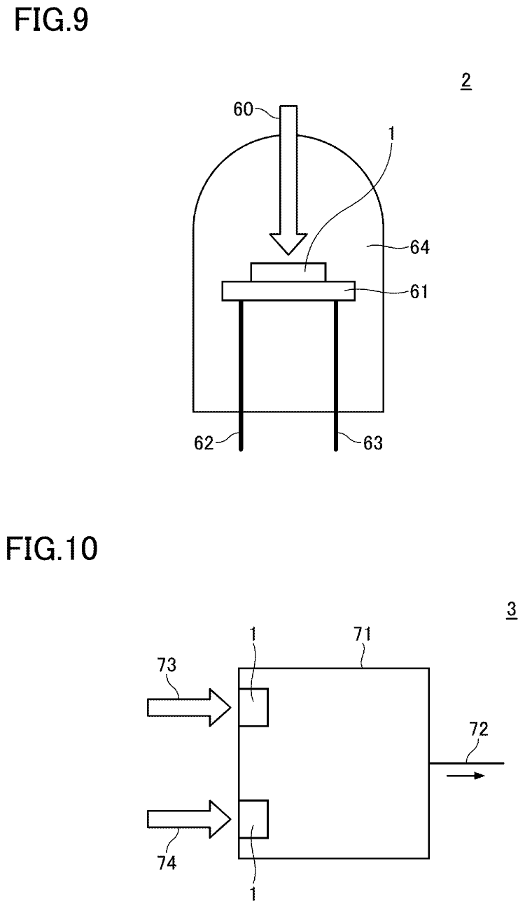

(Second Embodiment) The second embodiment will be described. The second embodiment relates to a photodetector including the semiconductor device 1 according to the first embodiment. FIG. 9 is a diagram illustrating the photodetector according to the second embodiment.

The photodetector 2 according to the second embodiment includes, as illustrated in FIG. 9, the semiconductor device 1, a substrate 61, a first lead terminal 62, a second lead terminal 63, and a package 64. The semiconductor device 1 is mounted on one surface of the substrate 61. The first lead terminal 62 and the second lead terminal 63 extend from the other surface of the substrate 61. The first lead terminal 62 is electrically connected to the first ohmic electrode 21 via the substrate 61, and the second lead terminal 63 is electrically connected to the second ohmic electrode 22 via the substrate 61. The substrate 61 and the semiconductor device 1 are housed in the package 64. The first lead terminal 62 and the second lead terminal 63 extend to the outside of the package 64. The package 64 is formed of a material that is transmissive to light 60 having a wavelength at which the semiconductor device 1 exhibits photoconductivity.

In the photodetector 2, a predetermined voltage is applied between the first lead terminal 62 and the second lead terminal 63, and the current flowing between the first lead terminal 62 and the second lead terminal 63 is measured. When positive photoconductivity appears in the semiconductor device 1, the photodetector 2 can detect that light having a wavelength of 800 nm or shorter, for example visible light, has been applied. When negative photoconductivity appears in the semiconductor device 1, the photodetector 2 can detect that light having a wavelength longer than 800 nm, for example infrared light, has been applied. Further, when light having a wavelength of 800 nm or shorter is applied, the degree of positive photoconductivity varies depending on the wavelength. Therefore, the wavelength can also be specified according to the degree of positive photoconductivity, that is, the degree of increase in current. Similarly, when light having a wavelength longer than 800 nm is applied, the degree of negative photoconductivity varies depending on the wavelength. Therefore, the wavelength can also be specified according to the degree of negative photoconductivity, that is, the degree of decrease in current.

(Third Embodiment) The third embodiment will be described. The third embodiment relates to an optoelectronic logic gate including the semiconductor device 1 according to the first embodiment. FIG. 10 is a diagram illustrating the optoelectronic logic gate according to the third embodiment.

The optoelectronic logic gate 3 according to the third embodiment includes, as illustrated in FIG. 10, a logic circuit 71 having two inputs and one output. The logic circuit 71 includes one semiconductor device 1 for each of the two inputs. Light 73 is applied onto one semiconductor device 1, and light 74 is applied onto the other semiconductor device 1. The logic circuit 71 outputs the result of logical operation from an output 72 in accordance with the outputs of the two semiconductor devices 1. Here, it is assumed that the semiconductor device 1 outputs “1” when visible light is applied and outputs “0” when infrared light is applied.

When the logic circuit 71 is an AND circuit, the logical operation illustrated in Table 1 below is performed.

TABLE 1

| Input light 73 | | | Input light 74 | | | Output 72 |
|---|---|---|---|---|---|---|
| | | Logical | | | Logical | Logical |
| Light | Photoconductivity | value | Light | Photoconductivity | value | value |
| Infrared | Negative | 0 | Infrared | Negative | 0 | 0 |
| light | | | light | | | |
| Visible | Positive | 1 | Infrared | Negative | 0 | 0 |
| light | | | light | | | |
| Infrared | Negative | 0 | Visible | Positive | 1 | 0 |
| light | | | light | | | |
| Visible | Positive | 1 | Visible | Positive | 1 | 1 |
| light | | | light | | | |

Further, when the logic circuit 71 is an XNOR circuit, the logical operation illustrated in Table 2 below is performed.

TABLE 2

| Input light 73 | | | Input light 74 | | | Output 72 |
|---|---|---|---|---|---|---|
| | | Logical | | | Logical | Logical |
| Light | Photoconductivity | value | Light | Photoconductivity | value | value |
| Infrared | Negative | 0 | Infrared | Negative | 0 | 1 |
| light | | | light | | | |
| Visible | Positive | 1 | Infrared | Negative | 0 | 0 |
| light | | | light | | | |
| Infrared | Negative | 0 | Visible | Positive | 1 | 0 |
| light | | | light | | | |
| Visible | Positive | 1 | Visible | Positive | 1 | 1 |
| light | | | light | | | |

In this manner, according to the optoelectronic logic gate 3, logical operations corresponding to the wavelengths of the input signals, namely the lights 73 and 74, can be performed. The logic circuit 71 may be a circuit other than an AND circuit or an XNOR circuit.

The semiconductor device 1 can also be used in an optoelectronic memory or a gas detector, in addition to the photodetector 2 and the optoelectronic logic gate 3.

Although preferred embodiments and the like have been described in detail above, the present disclosure is not limited to the above-described embodiments and the like, and various modifications and substitutions can be added to the above-described embodiments and the like without departing from the scope described in the claims.

All examples and conditional language recited herein are intended for pedagogical purposes to aid the reader in understanding the invention and the concepts contributed by the inventor to furthering the art, and are to be construed as being without limitation to such specifically recited examples and conditions, nor does the organization of such examples in the specification relate to a illustrating of the superiority and inferiority of the invention. Although the embodiment(s) of the present inventions have been described in detail, it should be understood that the various changes, substitutions, and alterations could be made hereto without departing from the spirit and scope of the invention.

What is claimed is:

1. A semiconductor device comprising:
a first barrier layer;
a second barrier layer disposed on the first barrier layer;
a channel layer disposed on the second barrier layer;
a first ohmic electrode electrically connected to the channel layer; and
a second ohmic electrode separated from the first ohmic electrode and electrically connected to the channel layer, wherein
the first barrier layer has a superlattice structure including a first nitride semiconductor layer containing Al and a second nitride semiconductor layer containing Ga,
the second barrier layer includes a third nitride semiconductor layer containing Al and Ga,
the channel layer includes a fourth nitride semiconductor layer containing Ga, and
a surface of the second barrier layer facing the channel layer has nitrogen polarity.

2. The semiconductor device according to claim 1, wherein the superlattice structure includes at least four pairs of the first nitride semiconductor layer and the second nitride semiconductor layer.

3. The semiconductor device according to claim 1, wherein the first nitride semiconductor layer is an AlN layer, and the second nitride semiconductor layer is a GaN layer.

4. The semiconductor device according to claim 1, wherein the third nitride semiconductor layer is an AlGaN layer, and the fourth nitride semiconductor layer is a GaN layer.

5. A photodetector comprising the semiconductor device according to claim 1.

6. An optoelectronic logic gate comprising the semiconductor device according to claim 1.

7. An optoelectronic memory comprising the semiconductor device according to claim 1.

8. A gas detector comprising the semiconductor device according to claim 1.

9. A method for manufacturing a semiconductor device, the method comprising:
forming a first barrier layer;
forming a second barrier layer on the first barrier layer;
forming a channel layer on the second barrier layer;
forming a first ohmic electrode electrically connected to the channel layer; and
forming a second ohmic electrode separated from the first ohmic electrode and electrically connected to the channel layer, wherein
the first barrier layer has a superlattice structure including a first nitride semiconductor layer containing Al and a second nitride semiconductor layer containing Ga,
the second barrier layer includes a third nitride semiconductor layer containing Al and Ga,
the channel layer includes a fourth nitride semiconductor layer containing Ga, and
a surface of the second barrier layer facing the channel layer has nitrogen polarity.
